(19) United States
(12) Patent Application Publication
(10) Pub. No.: US 2018/0105597 A1
(43) Pub. Date: Apr. 19, 2018

(54) TGFbeta Receptor II Antibodies

(71) Applicant: Eli Lilly and Company, Indianapolis, IN (US)

(72) Inventors: Douglas Bryan Burtrum, New York, NY (US); Kyla Elizabeth Driscoll, Rutherford, NJ (US)

(21) Appl. No.: 15/729,818

(22) Filed: Oct. 11, 2017

Related U.S. Application Data
(60) Provisional application No. 62/409,402, filed on Oct. 18, 2016.

(51) Int. Cl. C07K 16/28; A61K 39/395; A61K 45/06

(52) U.S. Cl. CPC C07K 16/2863; A61K 39/39541; A61K 45/06; A61K 2039/505; C07K 16/2866; C07K 2317/51; C07K 2317/515; C07K 16/2827

(57) ABSTRACT

The present invention relates to antibodies that bind TGFβ receptor II (TGFβRII), and may be useful for treating cancer alone and in combination with chemotherapy and other cancer therapeutics.

The present invention relates to the field of medicine. More particularly, the present invention relates to antibodies that bind TGFβ receptor II (TGFβRII), and may be useful for treating cancer alone and in combination with chemotherapy and other cancer therapeutics.

The transforming growth factor β (TGFβ) pathway has pleiotropic functions regulating cell growth, differentiation, apoptosis, motility and invasion, extracellular matrix production, angiogenesis, and immune response. TGFβ signaling deregulation is frequent in tumors and has crucial roles in tumor initiation, development, and metastasis.

The TGFβ pathway has differential effects at the early and late stages of carcinogenesis. In the pre-malignant state, TGFβ acts as a tumor suppressor, potently inhibiting the proliferation of cells. However, upon malignant progression, the tumor suppressor arm of the pathway is lost and cancer cells become resistant to the growth-inhibitory effects. Aggressive tumors are typically associated with high TGFβ levels, which in turn are closely associated with poor prognosis in various tumor types. At the tumor microenvironment level, the TGFβ pathway contributes to generate a favorable microenvironment for tumor growth and metastasis throughout all the steps of carcinogenesis.

The binding of TGFβ ligands to TGFβ RII is the first step to initiate activation of the TGFβ signaling pathway. Therefore, blockade of ligand binding to TGFβ RII could be an effective approach to inhibit the multitude of TGFβ pathway effects on cancer progression.

Anti-TGFβ RII antibodies are disclosed in WO 2010/053814 including a TGFβRII mAb called LY3022859. LY3022859 blocks the ectodomain of TGFβ RII, thereby preventing the formation of the ligand-receptor complex, and thus inhibiting receptor-mediated signaling. During a phase I study with LY3022859 it was discovered by Applicant as part of the present invention that dosing of LY3022859 resulted in rapid infusion-related reactions characterized by cytokine release syndrome (CRS).

Thus, anti-TGFβ RII antibodies are needed that avoid or reduce CRS in patients. Further, anti-TGFβ RII antibodies are needed that avoid or reduce CRS in patients and maintain activity comparable to LY3022859 against TGFβ RII as measured by in vitro assays.

For the TGFβ pathway, CRS has not been seen in human clinical trials with TGFβ compounds such as small molecule inhibitors of TGFβ RI or antibodies directed to TGFβ ligands. For the IgG1 backbone of LY3022859, CRS has not been seen with numerous clinically safe antibodies that share a similar IgG1 backbone.

Applicants identified mutations in the CH2 region of the heavy chain of LY3022859 that cause less cytokine release compared to LY3022859 in three in vitro cytokine release assays that are modified to be more predictive and specific than standard cytokine release assays for LY3022859 induced cytokine release. The identified mutations ablate binding to Fcγ receptor expressing cells and complement components in order to inhibit cross-linking by the Fab portion of the anti-TGFβ RII antibody to TGFβ RII-containing cells, and the Fc portion of the anti-TGFβ RII antibody to immune cells.

Accordingly, the present invention provides an antibody comprising two light chains (LC) and two heavy chains (HC), wherein the amino acid sequence of each LC is SEQ ID NO: 5, and the amino acid sequence of each HC is SEQ ID NO: 8. In some embodiments, the present invention provides an antibody comprising two light chains (LC) and two heavy chains (HC), wherein the amino acid sequence of each LC is SEQ ID NO: 5, and the amino acid sequence of each HC is SEQ ID NO: 4. These antibodies retain the TGFβ RII binding variable regions of LY3022859, which are provided herein as SEQ ID NO:2 and SEQ ID NO:3.

In further embodiments, the present invention provides an antibody that binds human TGFβ Receptor II (SEQ ID NO: 1), comprising two light chains and two heavy chains, wherein the amino acid sequence of each LC is SEQ ID NO: 5, and the amino acid sequence of each HC is SEQ ID NO: 4. In further embodiments, the present invention provides an antibody that binds human TGFβ Receptor II (SEQ ID NO: 1), comprising two light chains and two heavy chains, wherein the amino acid sequence of each LC is SEQ ID NO: 5, and the amino acid sequence of each HC is SEQ ID NO: 8.

In some embodiments, the present invention provides an antibody that binds human TGFβ RII (SEQ ID NO: 1), wherein the antibody prevents cross-linking of TGFβ RII expressing cells and Fcγ receptor expressing cells. In further embodiments, the present invention provides an antibody that binds human TGFβ Receptor II (SEQ ID NO: 1), comprising two light chains and two heavy chains, wherein the amino acid sequence of each LC is SEQ ID NO: 5, and the amino acid sequence of each HC is SEQ ID NO: 8, and wherein the antibody prevents cross-linking of TGFβ RII expressing cells and Fcγ receptor expressing cells. In further embodiments, the present invention provides an antibody that binds human TGFβ Receptor II (SEQ ID NO: 1), comprising two light chains and two heavy chains, wherein the amino acid sequence of each LC is SEQ ID NO: 5, and the amino acid sequence of each HC is SEQ ID NO: 4, and wherein the antibody prevents cross-linking of TGFβ RII expressing cells and Fcγ receptor expressing cells.

In an embodiment, the present invention provides a mammalian cell comprising a DNA molecule comprising a polynucleotide sequence encoding a polypeptide having the amino acid sequence of SEQ ID NO: 5 and a polynucleotide sequence encoding a polypeptide having the amino acid sequence of SEQ ID NO: 4, wherein the cell is capable of expressing an antibody comprising a light chain having the amino acid sequence of SEQ ID NO: 5 and a heavy chain having the amino acid sequence of SEQ ID NO: 4. In an embodiment, the present invention provides a mammalian cell comprising a DNA molecule comprising a polynucleotide sequence encoding a polypeptide having the amino acid sequence of SEQ ID NO: 5 and a polynucleotide sequence encoding a polypeptide having the amino acid sequence of SEQ ID NO: 8, wherein the cell is capable of expressing an antibody comprising a light chain having the amino acid sequence of SEQ ID NO: 5 and a heavy chain having the amino acid sequence of SEQ ID NO: 8.

In an embodiment, the present invention provides a mammalian cell comprising a first DNA molecule and a second DNA molecule, wherein the first DNA molecule comprises a polynucleotide sequence encoding a polypeptide having the amino acid sequence of SEQ ID NO: 5, and wherein the second DNA molecule comprises a polynucleotide sequence encoding a polypeptide having the amino acid sequence of SEQ ID NO: 4, wherein the cell is capable of expressing the antibody comprising a light chain having the amino acid sequence of SEQ ID NO: 5 and a heavy chain having the amino acid sequence of SEQ ID NO: 4. In an embodiment, the present invention provides a mammalian cell comprising a first DNA molecule and a second DNA molecule, wherein the first DNA molecule comprises a polynucleotide sequence encoding a polypeptide having the amino acid sequence of SEQ ID NO: 5, and wherein the second DNA molecule comprises a polynucleotide sequence encoding a polypeptide having the amino acid sequence of SEQ ID NO: 8, wherein the cell is capable of expressing the antibody comprising a light chain having the amino acid sequence of SEQ ID NO: 5 and a heavy chain having the amino acid sequence of SEQ ID NO: 8.

In an embodiment, the present invention provides a process for producing an antibody, comprising a light chain having an amino acid sequence of SEQ ID NO: 5 and a heavy chain having an amino acid sequence of SEQ ID NO: 4, comprising cultivating the mammalian cell of the present invention under conditions such that the antibody is expressed, and recovering the expressed antibody. In an embodiment, the present invention provides a process for producing an antibody, comprising a light chain having an amino acid sequence of SEQ ID NO: 5 and a heavy chain having an amino acid sequence of SEQ ID NO: 8, comprising cultivating the mammalian cell of the present invention under conditions such that the antibody is expressed, and recovering the expressed antibody.

In an embodiment, the present invention provides an antibody produced by a process of the present invention.

In an embodiment, the present invention provides a pharmaceutical composition, comprising an antibody of the present invention, and an acceptable carrier, diluent, or excipient.

In an embodiment, the present invention provides a method of treating fibrosis, comprising administering to a patient in need thereof, an effective amount of the antibody of the present invention.

In an embodiment, the present invention provides a method of treating cancer, comprising administering to a patient in need thereof, an effective amount of an antibody of the present invention. In a further embodiment, the present invention provides a method of treating cancer, comprising administering to a patient in need thereof, an effective amount of an antibody of the present invention, wherein the cancer is breast cancer, colon cancer, gastric cancer, glioblastoma, head and neck cancer, hepatocellular carcinoma, non-small cell lung cancer (NSCLC), small cell lung cancer (SCLC), melanoma, myelodysplastic syndrome, pancreatic cancer, prostate cancer, or renal cancer.

In a further embodiment, these methods comprise the administration of an effective amount of the antibody of the present invention in simultaneous, separate, or sequential combination with one or more anti-tumor agents, wherein the anti-tumor agents are selected from the list consisting of platinum-based antineoplastic drugs, taxanes, fluoropyrimidines, enzalutamide, abiraterone, sorafenib, IMC-GP100, abemaciclib, and ramucirumab.

In a further embodiment, these methods comprise the administration of an effective amount of the compound of the present invention in simultaneous, separate, or sequential combination with one or more immuno-oncology agents, wherein the immuno-oncology agents are selected from the list consisting of nivolumab, ipilimumab, pidilizumab, pembrolizumab, tremelimumab, urelumab, lirilumab, atezolizumab, and durvalumab.

In a further embodiment, these methods comprise the administration of an effective amount of the antibody of the present invention in simultaneous, separate, or sequential combination with an anti-PD-L1 antibody, wherein the anti-PD-L1 antibody is Antibody C, comprising two light chains and two heavy chains, wherein each light chain has the amino acid sequence given in SEQ ID NO: 10, and each heavy chain has the amino acid sequence given in SEQ ID NO: 9.

In a further embodiment, these methods comprise the administration of an effective amount of the antibody of the present invention in simultaneous, separate, or sequential combination with an anti-C SF-1R antibody, wherein the anti-C SF-1R antibody is Antibody D, comprising two light chains and two heavy chains, wherein each light chain has the amino acid sequence given in SEQ ID NO: 12, and each heavy chain has the amino acid sequence given in SEQ ID NO: 11.

In an embodiment, the present invention provides an antibody of the present invention, for use in therapy. In an embodiment, the present invention provides an antibody of the present invention, for use in the treatment of fibrosis.

In an embodiment, the present invention provides an antibody of the present invention, for use in the treatment of cancer. In a further embodiment, the present invention provides an antibody of the present invention, for use in the treatment of cancer, wherein the cancer is breast cancer, colon cancer, gastric cancer, glioblastoma, head and neck cancer, hepatocellular carcinoma, non-small cell lung cancer (NSCLC), small cell lung cancer (SCLC), melanoma, myelodysplastic syndrome, pancreatic cancer, prostate cancer, or renal cancer.

In a further embodiment, the present invention provides an antibody of the present invention, for use in the treatment of cancer, wherein the cancer is breast cancer. In a further embodiment, the present invention provides an antibody of the present invention, for use in the treatment of cancer, wherein the cancer is colon cancer. In a further embodiment, the present invention provides an antibody of the present invention, for use in the treatment of cancer, wherein the cancer is gastric cancer. In a further embodiment, the present invention provides an antibody of the present invention, for use in the treatment of cancer, wherein the cancer is glioblastoma. In a further embodiment, the present invention provides an antibody of the present invention, for use in the treatment of cancer, wherein the cancer is head and neck cancer. In a further embodiment, the present invention provides an antibody of the present invention, for use in the treatment of cancer, wherein the cancer is hepatocellular carcinoma. In a further embodiment, the present invention provides an antibody of the present invention, for use in the treatment of cancer, wherein the cancer is non-small cell lung cancer. In a further embodiment, the present invention provides an antibody of the present invention, for use in the treatment of cancer, wherein the cancer is small cell lung cancer. In a further embodiment, the present invention provides an antibody of the present invention, for use in the treatment of cancer, wherein the cancer is melanoma. In a further embodiment, the present invention provides an antibody of the present invention, for use in the treatment of cancer, wherein the cancer is myelodysplastic syndrome. In a further embodiment, the present invention provides an antibody of the present invention, for use in the treatment of cancer, wherein the cancer is pancreatic cancer. In a further embodiment, the present invention provides an antibody of the present invention, for use in the treatment of cancer, wherein the cancer is prostate cancer. In a further embodiment, the present invention provides an antibody of the present invention, for use in the treatment of cancer, wherein the cancer is renal cancer.

In a further embodiment, the present invention provides the antibody of the present invention for use in simultaneous, separate, or sequential combination with one or more anti-tumor agents selected from the group consisting of platinum-based antineoplastic drugs, taxanes, fluoropyrimidines, enzalutamide, abiraterone, sorafenib, IMC-GP100, abemaciclib, and ramucirumab.

In a further embodiment, the present invention provides the antibody of the present invention for use in simultaneous, separate, or sequential combination with one or more immuno-oncology agents selected from the group consisting of nivolumab, ipilimumab, pidilizumab, pembrolizumab, tremelimumab, urelumab, lirilumab, atezolizumab, and durvalumab, in the treatment of cancer.

In a further embodiment, the present invention provides the antibody of the present invention for use in simultaneous, separate, or sequential combination with an anti-PD-L1 antibody, wherein the anti-PD-L1 antibody is Antibody C, comprising two light chains and two heavy chains, wherein each light chain has the amino acid sequence given in SEQ ID NO: 10, and each heavy chain has the amino acid sequence given in SEQ ID NO: 9.

In a further embodiment, the present invention provides the antibody of the present invention for use in simultaneous, separate, or sequential combination with an anti-CSF-1R antibody, wherein the anti-CSF-1R antibody is Antibody D, comprising two light chains and two heavy chains, wherein each light chain has the amino acid sequence given in SEQ ID NO: 12, and each heavy chain has the amino acid sequence given in SEQ ID NO: 11.

In a further embodiment, the present invention provides the antibody of the present invention for use in the treatment of cancer in simultaneous, separate, or sequential combination with one or more of the following:
A.) One or more anti-tumor agents selected from the group consisting of platinum-based antineoplastic drugs, taxanes, fluoropyrimidines, enzalutamide, abiraterone, sorafenib, IMC-GP100, abemaciclib, and ramucirumab;
B.) One or more immuno-oncology agents selected from the group consisting of nivolumab, ipilimumab, pidilizumab, pembrolizumab, tremelimumab, urelumab, lirilumab, atezolizumab, and durvalumab;
C.) An anti-PD-L1 antibody, comprising two light chains and two heavy chains, wherein each light chain has the amino acid sequence given in SEQ ID NO: 10, and each heavy chain has the amino acid sequence given in SEQ ID NO: 9;
D.) Anti-CSF-1R antibody, comprising two light chains and two heavy chains, wherein each light chain has the amino acid sequence given in SEQ ID NO: 12, and each heavy chain has the amino acid sequence given in SEQ ID NO: 11.

In an embodiment, the present invention provides the use of an antibody of the present invention for the manufacture of a medicament for the treatment of fibrosis.

In an embodiment, the present invention provides the use of an antibody of the present invention for the manufacture of a medicament for the treatment of cancer. In a further embodiment, the present invention provides the use of an antibody of the present invention for the manufacture of a medicament for the treatment of cancer, wherein the cancer is breast cancer, colon cancer, gastric cancer, glioblastoma, head and neck cancer, hepatocellular carcinoma, non-small cell lung cancer (NSCLC), small cell lung cancer (SCLC), melanoma, myelodysplastic syndrome, pancreatic cancer, prostate cancer, or renal cancer.

In a further embodiment, the present invention provides the use of an antibody of the present invention in the manufacture of a medicament for the treatment of cancer wherein said medicament is to be administered simultaneously, separately, or sequentially with one or more anti-tumor agents selected from the group consisting of platinum-based antineoplastic drugs, taxanes, fluoropyrimidines, enzalutamide, abiraterone, sorafenib, IMC-GP100, abemaciclib, and ramucirumab.

In an embodiment, the present invention provides a pharmaceutical composition for use in treating fibrosis, comprising an effective amount of an antibody of the present invention.

In an embodiment, the present invention provides a pharmaceutical composition for use in treating cancer, comprising an effective amount of an antibody of the present invention. In a further embodiment, the present invention provides a pharmaceutical composition for use in treating cancer, comprising an effective amount of an antibody of the present invention, wherein the cancer is breast cancer, colon cancer, gastric cancer, glioblastoma, head and neck cancer, hepatocellular carcinoma, non-small cell lung cancer (NSCLC), small cell lung cancer (SCLC), melanoma, myelodysplastic syndrome, pancreatic cancer, prostate cancer, or renal cancer.

In a further embodiment, the present invention provides a pharmaceutical composition for use in treating cancer, comprising an effective amount of an antibody of the present invention, wherein the cancer is breast cancer. In a further embodiment, the present invention provides a pharmaceutical composition for use in treating cancer, comprising an effective amount of an antibody of the present invention, wherein the cancer is colon cancer. In a further embodiment, the present invention provides a pharmaceutical composition for use in treating cancer, comprising an effective amount of an antibody of the present invention, wherein the cancer is gastric cancer. In a further embodiment, the present invention provides a pharmaceutical composition for use in treating cancer, comprising an effective amount of an antibody of the present invention, wherein the cancer is glioblastoma. In a further embodiment, the present invention provides a pharmaceutical composition for use in treating cancer, comprising an effective amount of an antibody of the present invention, wherein the cancer is head and neck cancer. In a further embodiment, the present invention provides a pharmaceutical composition for use in treating cancer, comprising an effective amount of an antibody of the present invention, wherein the cancer is hepatocellular carcinoma. In a further embodiment, the present invention provides a pharmaceutical composition for use in treating cancer, comprising an effective amount of an antibody of the present invention, wherein the cancer is non-small cell lung cancer. In a further embodiment, the present invention provides a pharmaceutical composition for use in treating cancer, comprising an effective amount of an antibody of the present invention, wherein the cancer is small cell lung cancer. In a further embodiment, the present invention provides a pharmaceutical composition for use in treating cancer, comprising an effective amount of an antibody of the present invention, wherein the cancer is melanoma. In a further embodiment, the present invention provides a pharmaceutical composition for use in treating cancer, comprising an effective amount of an antibody of the present invention, wherein the cancer is myelodysplastic syndrome. In a further embodiment, the present invention provides a pharmaceutical composition for use in treating cancer, comprising an effective amount of an antibody of the present invention, wherein the cancer is pancreatic cancer. In a further embodiment, the present invention provides a pharmaceutical composition for use in treating cancer, comprising an effective amount of an antibody of the present invention, wherein the cancer is prostate cancer. In a further embodiment, the present invention provides a pharmaceutical composition for use in treating cancer, comprising an effective amount of an antibody of the present invention, wherein the cancer is renal cancer.

In a further embodiment, the present invention provides a pharmaceutical composition for use in treating cancer, wherein said pharmaceutical composition is administered in simultaneous, separate, or sequential combination with one or more anti-tumor agents, wherein the anti-tumor agents are selected from the list consisting of platinum-based antineoplastic drugs, taxanes, fluoropyrimidines, enzalutamide, abiraterone, sorafenib, IMC-GP100, abemaciclib, and ramucirumab.

In a further embodiment, the present invention provides a pharmaceutical composition for use in treating cancer, wherein said pharmaceutical composition is administered in simultaneous, separate, or sequential combination with one or more immuno-oncology agents selected from the group consisting of nivolumab, ipilimumab, pidilizumab, pembrolizumab, tremelimumab, urelumab, lirilumab, atezolizumab, and durvalumab.

In a further embodiment, the present invention provides a pharmaceutical composition for use in treating cancer, wherein said pharmaceutical composition is administered in simultaneous, separate, or sequential combination with an anti-PD-L1 antibody, wherein the anti-PD-L1 antibody is Antibody C, comprising two light chains and two heavy chains, wherein each light chain has the amino acid sequence given in SEQ ID NO: 10, and each heavy chain has the amino acid sequence given in SEQ ID NO: 9.

In a further embodiment, the present invention provides a pharmaceutical composition for use in treating cancer, wherein said pharmaceutical composition is administered in simultaneous, separate, or sequential combination with an anti-CSF-1R antibody, wherein the anti-CSF-1R antibody is Antibody D, comprising two light chains and two heavy chains, wherein each light chain has the amino acid sequence given in SEQ ID NO: 12, and each heavy chain has the amino acid sequence given in SEQ ID NO: 11.

An antibody of the present invention is an engineered, non-naturally occurring polypeptide complex. A DNA molecule of the present invention is a non-naturally occurring DNA molecule that comprises a polynucleotide sequence encoding a polypeptide having the amino acid sequence of one of the polypeptides in an antibody of the present invention.

The antibody of the present invention is an IgG type antibody and has “heavy” chains and “light” chains that are cross-linked via intra- and inter-chain disulfide bonds. Each heavy chain is comprised of an N-terminal HCVR and a heavy chain constant region (“HCCR”). Each light chain is comprised of a LCVR and a light chain constant region (“LCCR”). When expressed in certain biological systems, antibodies having native human Fc sequences are glycosylated in the Fc region. Typically, glycosylation occurs in the Fc region of the antibody at a highly conserved N-glycosylation site. N-glycans typically attach to asparagine. Antibodies may be glycosylated at other positions as well.

The antibody of the present invention is an antibody, wherein one of the heavy chains forms an inter-chain disulfide bond with one of the light chains, and the other heavy chain forms an inter-chain disulfide bond with the other light chain, and one of the heavy chains forms two inter-chain disulfide bonds with the other heavy chain.

Antibody A is a monoclonal antibody of the IgG1 subclass, modified by 5 CH2 amino acid mutations to ablate binding to Fcγ receptor expressing cells and complement components. Antibody B is modified by 3 CH2 amino acid mutations to ablate binding to Fcγ receptor expressing cells. Antibody A and Antibody B are composed of four polypeptide chains, two identical heavy (γ) chains consisting of 451 amino acids each, and two identical light (κ) chains consisting of 214 amino acids each. The four chains are held together by a combination of covalent (disulfide) and non-covalent bonds. There are 32 cysteine residues, and accordingly, 16 potential disulfide bonds per molecule. The heavy chain subunit contains one consensus sequence for N-linked glycosylation.

Antibody C is a recombinant IgG1 human monoclonal antibody targeting human PD-L1. Antibody C is an antibody, comprising two light chains and two heavy chains, wherein each light chain has the amino acid sequence given in SEQ ID NO: 10, and each heavy chain has the amino acid sequence given in SEQ ID NO: 9.

Antibody D is a recombinant IgG1 human monoclonal antibody targeting human CSF-1R. CSF-1R, as specified herein, includes either variation of CSF-1R as disclosed in SEQ ID NOS: 15 and 16 of U.S. Pat. No. 8,263,079. Antibody D and methods of making and using this antibody including for the treatment of neoplastic diseases such as solid tumors are disclosed in U.S. Pat. No. 8,263,079. Furthermore, clinical study of Antibody D is ongoing in two clinical trials (NCT01346358 and NCT02265536). Antibody D is an antibody, comprising two light chains and two heavy chains, wherein each light chain has the amino acid sequence given in SEQ ID NO: 12, and each heavy chain has the amino acid sequence given in SEQ ID NO: 11.

An isolated DNA encoding a HCVR region can be converted to a full-length heavy chain gene by operably linking the HCVR-encoding DNA to another DNA molecule encoding heavy chain constant regions. The sequences of human, as well as other mammalian, heavy chain constant region genes are known in the art. DNA fragments encompassing these regions can be obtained e.g., by standard PCR amplification.

An isolated DNA encoding a LCVR region may be converted to a full-length light chain gene by operably linking the LCVR-encoding DNA to another DNA molecule encoding a light chain constant region. The sequences of human, as well as other mammalian, light chain constant region genes are known in the art. DNA fragments encompassing these regions can be obtained by standard PCR amplification. The light chain constant region can be a kappa or lambda constant region.

The polynucleotides of the present invention will be expressed in a host cell after the sequences have been operably linked to an expression control sequence. The expression vectors are typically replicable in the host organisms either as episomes or as an integral part of the host chromosomal DNA. Commonly, expression vectors will contain selection markers, e.g., tetracycline, neomycin, and dihydrofolate reductase, to permit detection of those cells transformed with the desired DNA sequences.

The antibody of the present invention may readily be produced in mammalian cells such as CHO, NS0, HEK293 or COS cells. The host cells are cultured using techniques well known in the art.

The vectors containing the polynucleotide sequences of interest (e.g., the polynucleotides encoding the polypeptides of the antibody and expression control sequences) can be transferred into the host cell by well-known methods, which vary depending on the type of cellular host.

Various methods of protein purification may be employed and such methods are known in the art and described, for example, in Deutscher, Methods in Enzymology 182: 83-89 (1990) and Scopes, Protein Purification: Principles and Practice, 3rd Edition, Springer, N.Y. (1994).

The antibody of the present invention, or pharmaceutical compositions comprising the same, may be administered by parenteral routes (e.g., subcutaneous and intravenous). An antibody of the present invention may be administered to a patient alone with pharmaceutically acceptable carriers, diluents, or excipients in single or multiple doses. Pharmaceutical compositions of the present invention can be prepared by methods well known in the art (See, e.g., Remington: The Science and Practice of Pharmacy, L.V. Allen, Editor, 22nd Edition, Pharmaceutical Press, 2012) and comprise an antibody, as disclosed herein, and one or more pharmaceutically acceptable carriers, diluents, or excipients.

The term “treating” (or “treat” or “treatment”) refers to slowing, interrupting, arresting, alleviating, stopping, reducing, or reversing the progression or severity of an existing symptom, disorder, condition, or disease.

Abemaciclib is the International Nonproprietary Name for the pharmaceutical substance with the IUPAC name being N-{5-[(4-ETHYLPIPERAZIN-1-YL)METHYL]PYRIDIN-2-YL}-5-FLUORO-4-[4-FLUORO-2-METHYL-1-(1-METHYLETHYL)-1H-BENZIMIDAZOL-6-YL]PYRIMIDIN-2-AMINE. Abemaciclib has the CAS number of 1231929-97-7 and it has the structure

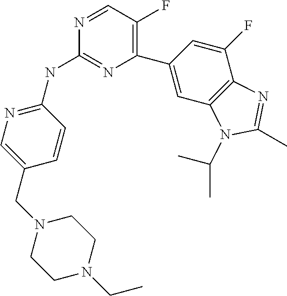 [SMILES: CCN1CCN(Cc2ccc(Nc3ncc(F)c(-c4cc(F)c5nc(C)n(C(C)C)c5c4)n3)nc2)CC1]

Ramucirumab is the International Nonproprietary Name for the pharmaceutical substance of Immunoglobulin G1, anti-(human vascular endothelial growth factor receptor 2 (fetal liver kinase 1, kinase insert domain receptor, protein-tyrosine kinase receptor Flk-1, CD309) extracellular domain); human monoclonal IMC-1121B (125-leucine(CH19-F>L))gamma1 heavy chain (219-214′)-disulfide with human monoclonal IMC-1121B kappa light chain dimer (225-225″:228-228″)-bisdisulfide. Ramucirumab has the CAS number of 947687-13-0.

Platinum-based anti-neoplastic drugs are chemotherapy drugs that contain a coordination complex with platinum, such as cisplatin, carboplatin, oxaliplatin, pyriplatin, and phenanthriplatin.

Taxanes are chemotherapy drugs that are diterpenes, such as paclitaxel, docetaxel, and cabazitaxel.

Fluoropyrimidines are chemotherapy drugs is a type of antimetabolite, such as capecitabine, floxuridine, and fluorouracil (5-FU).

“Binds” as used herein in reference to the affinity of an antibody for human TGFβRII is intended to mean, unless indicated otherwise, a K_{D} of less than about 1×10⁻⁶ M, preferably, less than about 1×10⁻⁹M as determined by common methods known in the art, including by use of a surface plasmon resonance (SPR) biosensor at 37° C. essentially as described herein.

“Effective amount” means the amount of an antibody of the present invention or pharmaceutical composition comprising an antibody of the present invention that will elicit the biological or medical response of or desired therapeutic effect on a tissue, system, animal, mammal or human that is being sought by the researcher, medical doctor, or other clinician. An effective amount of the antibody may vary according to factors such as the disease state, age, sex, and weight of the individual, and the ability of the antibody to elicit a desired response in the individual. An effective amount is also one in which any toxic or detrimental effect of the antibody is outweighed by the therapeutically beneficial effects.

This invention is further illustrated by the following non-limiting example.

### EXAMPLE 1: ANTIBODY EXPRESSION AND PURIFICATION

The polypeptides of the variable region of the heavy chain and light chain, the complete heavy chain and light chain amino acid sequences of Antibody A, and the nucleotide sequences encoding the same, are listed below in the section entitled “Amino Acid and Nucleotide Sequences.” In addition, the SEQ ID NOs for the light chain, heavy chain, light chain variable region, and heavy chain variable region of Antibody A are shown in Table 1.

The antibodies of the present invention, including, but not limited to, Antibody A can be made and purified essentially as follows. An appropriate host cell, such as HEK 293 or CHO, can be either transiently or stably transfected with an expression system for secreting antibodies using an optimal predetermined HC:LC vector ratio or a single vector system encoding both HC and LC. Secreted antibody may be purified from clarified medium using any of many commonly-used techniques. For example, the medium may be conveniently applied to a Mab Select column (GE Healthcare), or KappaSelect column (GE Healthcare) that has been equilibrated with a compatible buffer, such as phosphate buffered saline (pH 7.4). The column may be washed to remove nonspecific binding components. The bound antibody may be eluted, for example, by pH gradient (such as 20 mM Tris buffer pH 7 to 10 mM sodium citrate buffer pH 3.0, or phosphate buffered saline pH 7.4 to 100 mM glycine buffer pH 3.0). Antibody fractions may be detected, such as by SDS-PAGE, and then may be pooled. The antibody may be concentrated and/or sterile filtered using common techniques. Soluble aggregate and multimers may be effectively removed by common techniques, including size exclusion, hydrophobic interaction, ion exchange, multimodal, or hydroxyapatite chromatography. The purity of the antibody after these chromatography steps is greater than 95%. The product may be immediately frozen at −70° C. or may be lyophilized.

TABLE 1

SEQ ID NOs

| | Antibody A | Antibody B |
|---|---|---|
| HCVR | 2 | 2 |
| LCVR | 3 | 3 |
| Heavy chain | 4 | 8 |
| Light chain | 5 | 5 |

### Assays

### Cytokine Release Assays

To mimic the CRS seen with LY3022859 in patients, Applicants developed cytokine release assays, described below, that could detect cytokine release for LY3022859 in vitro: 1) wet-coated plate-bound whole blood assay, 2) wet-coated plate-bound leukocyte assay, and 3) air-dried plate-bound leukocyte assay. One key finding in assay development was that the antibody must be plate-bound in order to reveal LY3022859-mediated cytokine release; soluble assays did not consistently predict LY3022859 cytokine release.

These three assays were used to test if cytokine release was reduced for Antibody A compared to LY3022859.

### 1. Wet-Coated Plate-Bound Whole Blood Assay

The potential for LY3022859 and Antibody A to elicit cytokine release from human peripheral blood cells was tested in a wet-coated plate-bound whole blood assay.

Fresh unstimulated whole blood samples from healthy donors was added to tissue-culture plates pre-coated with test antibodies or control antibodies at 10 μg/ml in PBS. Coating antibody at 2 mL/well and assuming 100% efficiency in binding results in 20 μg antibody per well in the wet-coated plate-bound whole blood assay. Plates were incubated 16-20 hours at 37° C. before cytokines were measured in cell culture supernatants using a Luminex platform. The positive control was LPS (500 ng/mL; 1000 ng per well). The negative controls (10 μg/mL; 20 μg/well) were a hIgG1 isotype antibody and an internal control antibody against a different target but with the same heavy chain constant region sequence as LY3022859. The internal control antibody had been tested in a Phase I study with no CRS observed in patients.

LY3022859 was compared to Antibody A at conditions where LY3022859 cytokine release on average across donors (typically 4 donors per experiment) was significantly higher than the two negative control antibodies.

Response to LY3022859 and Antibody A was analyzed via 1-way ANOVA (n=4 donors). Results for Table 2 were organized first by cytokines where LY3022859 produced significantly higher than baseline level and then by cytokines where LY3022859 produced significantly higher than human IgG and 20D7S levels. Results are presented in Table 2 as p-values and fold changes with 95% confidence intervals for cytokine release with LY3022859 compared to Antibody A. Confidence intervals (CI) are calculated to describe the range of activity observed for a particular cytokine across the donors in the experiment.

In experiments performed essentially as described in this assay, incubation of whole blood with wet-coated plate-bound LY3022859 resulted in cytokine levels significantly above baseline for a number of analytes. Significantly higher levels of one or more cytokines were observed with LY3022859 in 25 of 34 tested donors. As shown in Table 2, lower or equivalent levels of cytokines were found for Antibody A in those conditions where cytokine release for LY3022859 was significantly higher than controls. Table 2 summarizes cytokines released in cultures induced by LY3022859, but not Antibody A or control antibodies, highlighting p-values and the fold change difference between LY3022859 over Antibody A.

TABLE 2
Summary of statistically significant cytokines in whole blood after
incubation with LY3022859 versus Antibody A
in wet-coated plate-bound assay

| | | | | |
|---|---|---|---|---|
| Analyte | p-value | Fold-change | Lower CI | Upper CI |

Experiment A (n = 4)

| | | | | |
|---|---|---|---|---|
| PDGF-aa | 0.0010 | 1.394 | 1.143 | 1.700 |
| IL-8 | 0.0122 | 1.425 | 1.077 | 1.885 |
| IL-6 | 0.0140 | 2.330 | 1.190 | 4.561 |
| RANTES | 0.0147 | 1.635 | 1.097 | 2.438 |
| IL-1a | 0.0320 | 1.825 | 1.050 | 3.171 |
| IL-10 | 0.0499 | 1.500 | 0.995 | 2.262 |
| MCP-1 | 0.0534 | 1.333 | 0.992 | 1.791 |

Experiment B (n = 4)

| | | | | |
|---|---|---|---|---|
| MIP-1a | 0.0000 | 10.174 | 3.814 | 27.140 |
| MIP-1b | 0.0001 | 3.013 | 1.725 | 5.265 |
| TNFa | 0.0003 | 3.468 | 1.803 | 6.669 |
| IL-6 | 0.0006 | 5.116 | 2.069 | 12.651 |
| IL-10 | 0.0061 | 2.290 | 1.265 | 4.145 |
| GRO | 0.0067 | 4.613 | 1.523 | 13.973 |
| GM-CSF | 0.0156 | 1.463 | 1.089 | 1.965 |
| IL-8 | 0.0708 | 2.402 | 0.917 | 6.291 |
| IL-1RA | 0.1465 | 1.490 | 0.864 | 2.569 |
| EOTAXIN | 0.5679 | 1.143 | 0.723 | 1.808 |

### 2. Wet-Coated Plate-Bound Leukocyte Cytokine Release Assay

The potential for LY3022859 and Antibody A to elicit cytokine release from human peripheral blood cells was tested in a wet-coated plate-bound leukocyte cytokine release assay.

Leukocytes from healthy donors were tested in a wet-coated plate-bound cytokine release assay. Leukocytes were obtained from whole blood from healthy donors by high density Ficoll separation. Isolated leukocytes were incubated with plate-bound LY3022859, Antibody A or control antibodies for 16-20 hours, at a fixed concentration of 10 μg/mL for 6-well plates and 20 μg/mL or over a broad titration range from 20 to 2.5 μg/mL when 96 well plates were applied. Coating antibody at 2 mL/well or 200 μl/well and assuming 100% efficiency in binding results in 20 μg antibody per well or in a range of 0.50-4 μg antibody per well for 6-well plates and 96-well plates, respectively. The negative controls were IgG1-effector null and the internal control antibody 20D7S at 10 or 20 μg/mL depending on the plate used (2-4 μg/well). The positive control was LPS (500 ng/mL; 100 ng).

Response to LY3022859 and Antibody A was analyzed via 1-way ANOVA. Data was analyzed as an average across donors included in the experiment (typically 4 donors). Results were organized first by cytokines where LY3022859 produced significantly higher than baseline level and then by cytokines where LY3022859 produced significantly higher than human IgG and 20D7S levels. Results were presented as p-values and fold changes with 95% confidence intervals for cytokine release with LY3022859 compared to Antibody A. Confidence intervals (CI) are calculated to describe the range of activity observed for a particular cytokine across the donors in the experiment.

In experiments performed essentially as described in this assay, incubation of leukocytes with wet-coated plate-bound LY3022859 resulted in cytokine levels significantly above baseline for a number of analytes as observed for whole blood. In conditions where LY3022859 fold change cytokine release on average across donors for each treatment in a particular experiment was significantly higher than baseline and IgG/20D7S, Antibody A was always lower or at the same level as LY3022859. Table 3 summarizes cytokines released in cultures induced by LY3022859, but not Antibody A or control antibodies, highlighting p-values and the fold change difference between LY3022859 over Antibody A.

TABLE 3
Summary of statistically significant cytokines in leukocytes
after incubation with LY3022859 versus Antibody A
in wet-coated plate-bound assay

| | | | | |
|---|---|---|---|---|
| Analyte | p-value | Fold change | Lower CI | Upper CI |

Experiment A (n = 4)

| | | | | |
|---|---|---|---|---|
| MCP-3 | 0.0013 | 1.544 | 1.185 | 2.010 |
| TNFa | 0.0433 | 1.845 | 1.017 | 3.346 |
| IL-1b | 0.2294 | 1.357 | 0.820 | 2.244 |
| IL-6 | 0.3356 | 1.257 | 0.784 | 2.014 |

Experiment B (n = 4)

| | | | | |
|---|---|---|---|---|
| GM-CSF | 0.0000 | 26.906 | 12.050 | 60.082 |
| TNFa | 0.0000 | 9.542 | 4.735 | 19.229 |
| IL-1b | 0.0000 | 19.539 | 7.672 | 49.758 |
| IL-1a | 0.0000 | 4.866 | 2.641 | 8.965 |
| IL-6 | 0.0000 | 8.743 | 3.331 | 22.944 |
| IL-10 | 0.0001 | 3.869 | 1.948 | 7.685 |
| G-CSF | 0.0002 | 6.754 | 2.564 | 17.792 |
| MIP-1b | 0.0006 | 5.040 | 2.018 | 12.584 |

### 3. Air-Dried Plate-Bound Leucocyte Assay

The potential for LY3022859 and Antibody A to elicit cytokine release from human peripheral blood cells is tested in an air-dried plate-bound leucocyte assay.

Leukocytes from healthy donors were tested in an air-dried plate-bound cytokine release assay. Leukocytes were obtained from whole blood from healthy donors by high density Ficoll separation. For the air-dried assay, plates were dried in a tissue culture hood overnight with antibodies diluted in PBS in a dose titration range. The therapeutic anti-CD28 antibody TGN1412, which had cytokine release syndrome in patients in a clinical trial, was used as a positive control along with LPS according to Stebbings et al., 2016.

Isolated leukocytes were incubated with air-dried plate-bound LY3022859, Antibody A or control antibodies for 16-20 hours. Plates were coated with TGN1412 (3-20 μg/mL), LY3022859 (20 μg/mL) and Antibody A (20 μg/mL). Positive controls were LPS (500 ng/mL). IgG and 20D7S were used as negative controls (20 μg/mL). Background are shown as lower limits of detection values calculated according to standard curve on the plate. Coating antibody at 200 μl/well and assuming 100% efficiency in binding results in 4 μg antibody per well.

In experiments performed essentially as described in this assay, certain cytokines showed higher than baseline and control antibody expression upon 16-20 hours incubation with LY3022859. The cytokine profile of LY3022859 in this assay was similar to the one observed in the wet-coated plate-bound assays. The therapeutic anti-CD28 antibody TGN1412 showed a different cytokine trend than LY3022859. The cytokine elevation seen with LY3022859 was absent with Antibody A treated cells.

### Effector Function Analysis

Antibody A was developed to reduce cross-linking mediated by the TGFbeta receptor II antibody leading to bridging TGFbRII expressing cells and immune cells while still maintaining the binding to TGFbeta receptor II of LY3022859. This cross-linking can be due to Fc mediated binding by the antibody to Fc gamma and cross-linking to a TGFβ RII expressing cells via the Fab, either in cis or in trans.

To confirm the inability of Antibody A to cross-link, the absence of binding to human Fc-gamma (Fcγ) receptors and C1q in solid phase binding ELISA assays was tested. Further, the absence of induction of antibody-dependent cellular cytotoxicity (ADCC) or complement-dependent cytotoxicity (CDC) response in cell-based in vitro assays was tested.

### Antibody Dependent Cell Cytotoxicity (ADCC)

ADCC is characterized by the killing of antibody coated target cells by immune effector cells with Fc receptors that recognize the constant region of the bound antibody. ADCC is typically induced by IgG1 antibodies.

LY3022859 and Antibody A were tested for binding to human Fc-gamma receptors in solid phase binding assays. Receptor protein (50 ng/well) was added to the wells of a Meso Scale standard bare plate in PBS. After overnight incubation at 4° C., plates were washed in PBS/0.05% Tween-20 3 times and subsequently blocked for 1-2 hours at room temperature with 150 μL/well 5% MSD Blocker in PBS/Tween-20. The plates were washed three times in PBS/0.05% Tween-20. Stock solutions of all antibodies were prepared at 0.5 mg/mL in 1% MSD blocker A in PBS-Tween and serially diluted 1:3. The indicated antibodies were added to each reaction well. Recombinant protein and antibodies were incubated for 2 hours at room temperature and then the plates were washed 2 times with PBS/0.05% Tween. Secondary antibody was added to each well. After one hour at room temperature with agitation followed by 40 mins at room temperature, the plates were washed 2 times with PBS/0.05% Tween-20. For detection, 150 μL of a 1× Read Buffer (MSD cat # R92TC-1, 4×) were added to each well. The plate was then read using a Sector Imager 2400.

In experiments performed essentially as described, LY3022859 demonstrated positive binding to all three Fcγ-receptors. In contrast, no binding of Antibody A was detected for any of the receptors tested at concentrations of antibody up to 2000 nM, including the high affinity V158 form of FcγR3a (Table 4).

In order to evaluate the potential for LY3022859 and Antibody A to mediate ADCC, they were tested in a Jurkat-FcγRIIIa Reporter Gene Assay using TGFβ RII positive cells as target cells (Table 5).

Target cells were washed in PBS and seeded in 96-well flat-bottom plates at 1×10⁴ cells/50 μL/well in 0.5% BSA in RPMI 1640. Plates were incubated at 37° C. in a 5% CO2 humidified incubator overnight. Jurkat-FcγRIIIa (V158) cells were collected, spun down and plated in T150 flasks in assay buffer overnight. Anti-TGFβ RII antibodies (IgG1 and IgG1 effector null) as well as Rituxan/Rituxan effector null were added in triplicate at 30 nM/50 μL/well to the wells containing the cells indicated above. Antibodies were titrated followed by 0.5-hour incubation on ice to prevent internalization. Jurkat-FcγRIIIa (V158) cells were collected, washed in PBS, and added at an E:T ratio of 24:1 at 50 μL/well followed by a 5-hour incubation in a 37° C.-5% CO2 humidified incubator. Plates were removed and left at room temperature for 15 minutes. Luciferase reagent was added at 100 μL/well and luminescence read on a Luminometer. WIL2-S cells and Rituxan-IgG1/IgG1-effector null antibodies were treated as assay control compounds.

In experiments performed essentially as described, LY3022859 displayed weak ADCC activity. No ADCC response was detected for Antibody A.

TABLE 4
Fcγ Receptor Binding Analyses of Antibody A and LY3022859

| | FcγR1 | FcγR2a(H) | FcγR3a (V) |
|---|---|---|---|
| | EC₅₀ (nM) | EC₅₀ (nM) | EC₅₀ (nM) |
| LY3022859 | 0.58 nM | 48 nM | 5.7 nM |
| Antibody A | >2000 | >2000 | >2000 |

Non-linear fit variable slope, n = 2, SEM. Data analyzed and plotted using GraphPad Prism.

### Complement Dependent Cytotoxicity (CDC)

CDC is another mechanism by which therapeutic antibodies can exert specific target cell lysis through activation of the complement system. CDC works by recruiting complement components in the serum to cells that are opsonized by antibodies. This effector function is mediated by binding of the Fc region of the antibody to the C1q complex of the complement system, resulting in activation of the complement cascade and generation of the membrane attack complex, leading to lysis of the antibody-bound cells.

The efficacy of antibodies to activate an attack mediated by the complement immune system to kill specific target cells can be measured in CDC assays. In order to evaluate the potential of LY3022859 and Antibody A to mediate CDC, Antibody A was tested using TGFβ RII positive cells as target cells (Table 5).

TABLE 5
Cell Lines for ADCC and CDC Assays

| Cell Line ID | Cell Line Indication | Comments |
|---|---|---|
| TGFβRII- | Human Embryonic Kidney | Positive (overexpressing) |
| HEK293 | | control cell line for |
| | | TGFβRII |
| HEK293 | Human Embryonic Kidney | Positive control cell line |
| | | for TGFβRII |
| MDA-MB-231 | Breast Cancer | Target cell line, |
| | | TGFβRII+ |
| BxPC-3 | Pancreatic Adenocarcinoma | Target cell line, |
| | | TGFβRII+ |
| WIL2-S | Spleen B cell lymphoblast | Assay positive control |
| Jurkat_FcγRIIIa | T cell line | NFAT luciferease |
| (V158) | | reporter line |

CDC activity was determined by measuring the percentage of live target cells after treatment with increasing concentrations of Antibody A, LY3022859, or control hIgG in the presence of human serum. Alamar Blue® was added after the treatment period and cell viability measured with a spectrophotometer. Target cells (Table 5) were washed in PBS and seeded in 96-well flat-bottom plates at 2.5×10⁴ cells/50 μL/well in RPMI 1640 without phenol red, 10% FBS with 0.1% BSA, 1-fold MEM NEAA, 1-fold GlutaMax™, 1-fold Sodium pyruvate, 1% Penicillin/Streptomycin, and 25 mM HEPES. Plates were incubated at 37° C. in a 5% CO2 humidified incubator overnight. Target cells for positive control (Wil2-S) were washed in PBS and seeded in 96-well flat-bottom plates in RPMI 1640 without phenol red, 10% FBS with 0.1% BSA, 1-fold MEM NEAA, 1-fold GlutaMax™, 1-fold Sodium pyruvate, 1% Penicillin/Streptomycin, and 25 mM HEPES. Plates were incubated at 37° C. in a 5% CO2 humidified incubator. Rituxan (positive control), Rituxan IgG1-effector null (negative control), and anti-TGFβ RII antibodies were added in duplicate to the plate. Antibodies may be titrated 1:5. Plates were incubated for 0.5-hour at a 37° C. in a 5% CO2 humidified incubator. Human complement was reconstituted and diluted in 5-mL of assay buffer (1:5). 50 μL of human complement was added to the assay plate. Plates were incubated for 1 hour at a 37° C. in a 5% CO2 humidified incubator. Alamar Blue® reagent was added at 16 μL/well (10% of final volume) to the plate, followed by a 22-hour incubation in a humidified 37° C. incubator. Plates were removed and equilibrated to room temperature for 5 minutes. Fluorescence was read on SpectraMax M5e at (Ex:560, Em:590, Auto cutoff On:590, top read).

In experiments performed essentially as described, as shown in Table 6, neither Antibody A or LY3022859 mediated a CDC response in any of the cell lines from Table 5.

The ability of Antibody A to promote CDC was further evaluated by measuring its binding to human C1q in a solid phase binding ELISA. Microtiter plates were coated with antibody of interest in duplicate with 50 μL/well at concentrations ranging from 50-0.078 μg/mL in PBS. After an overnight incubation at 4° C., 300 μl of casein buffer was added and plates incubated for 2 hours at room temperature. The plates were washed to remove unbound antibody, and then C1q protein (0.5 μg) added per well in casein blocking buffer. After 2 hours at room temperature, the plates were washed 3 times with PBS-Tween-20. Secondary antibody (sheep anti-human C1q-HRP) was added to each well. After 1 h at room temperature, the plates were washed 3 times with PBS-Tween-20. TMB substrate was added to each well and the plates incubated at room temperature for 20 min. The reaction may be stopped with the addition of stop solution and absorbance read at 450 nM using a microplate reader.

In experiments performed essentially as described, LY3022859 demonstrates binding to C1q with a calculated EC₅₀ of 19 nM. In contrast, Antibody A displays significantly weaker binding in both potency and magnitude of binding. An EC₅₀ value for Antibody A could not be calculated due to the lack of a saturable binding in the concentration range tested.

### SUMMARY

Together these results confirm that Antibody A is incapable of promoting ADCC and CDC in the assays tested, and so also has reduced capacity to promote cross-linking of TGFβ RII expressing cells and immune cells.

TABLE 6
Assessment of CDC Activity with ANTIBODY A

| | WIL2S | TGFRβII- | MDA-MB- | | |
|---|---|---|---|---|---|
| | EC₅₀ | HEK | 231 | BxPC3 | HEK293 |
| | (nM) | EC₅₀ (nM) | EC₅₀ (nM) | EC₅₀ (nM) | EC₅₀ (nM) |
| LY3022859 | NT | >200 nM | >200 nM | >200 nM | >200 nM |
| Antibody A | NT | >200 nM | >200 nM | >200 nM | >200 nM |
| Rituximab | 3.3 nM | NT | NT | NT | NT |
| Rituximab- | >200 | NT | NT | NT | NT |
| EN | nM | | | | |

NT—not tested

### Binding and Blocking Characteristics of Antibody A and LY3022859

In order to determine that Antibody A has comparable activity to LY3022859, binding to human TGFβ RII was compared by ELISA to determine the drug concentration that produces 50% of maximal response (EC₅₀) and by Biacore to obtain the drug concentration required for saturation of 50% of receptors (K_{D}).

For binding assays, huTGFβ RIIFc was coated on microplates. Serial dilutions of Antibody A and LY3022859 were independently incubated for 1 h in huTGFβ RIIFc-coated plates. After washing, plates were incubated with HRP-labelled goat anti-rat Fab detection antibody and subsequently TMB peroxidase substrate was added to the wells for color development. The values of absorbance at 405 nm were read on a microtiter plate reader (Molecular Devices Corp). EC₅₀ of the antibodies was analyzed using GraphPad Prism.

Affinities of Antibody A and LY3022859 for human TGFβ RIIFc were determined by Surface Plasmon Resonance (SPR) Technology by immobilizing the recombinant extracellular domain of the TGFβ RII-Fc onto the sensor surface at a low density. The association (kon) and dissociation (koff) rates were determined using BIAevaluation 2.1 software.

For blocking assay, TGFβ ligands (TGFb1, TGFb2, and TGFb3) were coated on microplates. A serial dilution of purified antibodies was incubated with TGFβ RII AP for 1 h in TGFβ-coated plates. After wash, p-nitrophenyl phosphate substrate was added to the wells for color development. The values of absorbance at 405 nm were read on a microtiter plate reader (Molecular Devices Corp) for the quantification of TGFβ RII binding to TGFβ. IC50 of the antibodies was analyzed using GraphPad Prism.

In experiments performed essentially as described, both Antibody A and LY3022859 showed strong binding affinity to human TGFβRII with EC₅₀ values of 0.277 nM and 0.306 nM, respectively, and K_{D} values of 22.2 pM and 10.6 pM, respectively. Similarly, both Antibody A and LY3022859 effectively blocked the binding of TGFβ ligand 1, 2, and 3 to TGFβ RII in ligand blocking ELISA assays with IC₅₀ values of 4.83 nM, 4.40 nM, and 4.36 nM, and 5.48 nM, 4.45, and 4.41 nM, respectively. Thus, Antibody A and LY3022859 show comparable binding and blocking properties for these assays.

### Efficacy Models

TGFβ signaling plays important pleiotropic roles in the tumor microenvironment. These include both tumor cell intrinsic and tumor cell extrinsic activity. To study the tumor cell intrinsic activity, xenograft models of human tumors are evaluated. One model, the BxPC3 model of human pancreatic cancer, was used to test Antibody A activity and compare to LY3022859 activity.

### BxPC3 Xenograft Model of Human Pancreatic Cancer

The anti-tumor efficacy of Antibody A and LY3022859 were compared in a xenograft model (BxPC-3) of human pancreatic carcinoma. Because Antibody A does not interact with mouse cells, this study assesses the direct anti-tumor effects of Antibody A-mediated inhibition of the TGFβ pathway in tumor cells in vivo.

To establish xenograft cancer models, a suspension of human tumor cells (BxPC-3) was injected SC on the flank of immune-compromised nu/nu mice. Once tumor volume reached ˜220 mm³ (day 6 post tumor challenge) treatment intraperitoneally (ip) with test compounds (40 mg/kg) or control (huIgG1) was commenced and continued three times per week for the duration of the study until tumors the control group reached ˜2000 mm³. Tumor volume data was analyzed through day 45 with two-way repeated measures analysis of variance by time and treatment using the MIXED procedures in SAS software (Version 9.2).

In experiments performed essentially as described, Antibody A significantly inhibited subcutaneous BxPC-3 pancreatic tumor growth with a T/C % of 24% (calculated as a treated over control tumor volume ratio T/C %) (p<0.001). LY3022859 was also efficacious in inhibiting tumor growth with a T/C % of 48% (p=0.002). Antibody A showed a trend for better efficacy than LY3022859 at the end of the study, but this difference did not reach statistical significance (p=0.28). Treatments were well-tolerated as monitored by body weight. The BxPC-3 model represents a preclinical model of aggressive human disease, and is therefore of interest as potential representation of indications proposed for Antibody A.

Amino Acid and Nucleotide Sequences
(human TGFbeta Receptor II)
SEQ ID NO: 1
MGRGLLRGLWPLHIVLWTRIASTIPPHVQKSVNNDMIVTDNNGAVKFPQL
CKFCDVRFSTCDNQKSCMSNCSITSICEKPQEVCVAVWRKNDENITLETV
CHDPKLPYHDFILEDAASPKCIMKEKKKPGETFFMCSCSSDECNDNIIFS
EEYNTSNPDLLLVIFQVTGISLLPPLGVAISVIIIFYCYRVNRQQKLSST
WETGKTRKLMEFSEHCAIILEDDRSDISSTCANNINHNTELLPIELDTLV
GKGRFAEVYKAKLKQNTSEQFETVAVKIFPYEEYASWKTEKDIFSDINLK
HENILQFLTAEERKTELGKQYWLITAFHAKGNLQEYLTRHVISWEDLRKL
GSSLARGIAHLHSDHTPCGRPKMPIVHRDLKSSNILVKNDLTCCLCDFGL
SLRLDPTLSVDDLANSGQVGTARYMAPEVLESRMNLENVESFKQTDVYSM
ALVLWEMTSRCNAVGEVKDYEPPFGSKVREHPCVESMKDNVLRDRGRPEI
PSFWLNHQGIQMVCETLTECWDHDPEARLTAQCVAERFSELEHLDRLSGR
SCSEEKIPEDGSLNTTK
(HCVR of Antibody A)
SEQ ID NO: 2
QLQVQESGPGLVKPSETLSLTCTVSGGSISNSYFSWGWIRQPPGKGLEWI
GSFYYGEKTYYNPSLKSRATISIDTSKSQFSLKLSSVTAADTAVYYCPRG
PTMIRGVIDSWGQGTLVTVSS
(LCVR of Antibody A)
SEQ ID NO: 3
EIVLTQSPATLSLSPGERATLSCRASQSVRSYLAWYQQKPGQAPRLLIYD
ASNRATGIPARFSGSGSGTDFTLTISSLEPEDFAVYYCQQRSNWPPTFGQ
GTKVEIK
(HC of Antibody A)
SEQ ID NO: 4
QLQVQESGPGLVKPSETLSLTCTVSGGSISNSYFSWGWIRQPPGKGLEWI
GSFYYGEKTYYNPSLKSRATISIDTSKSQFSLKLSSVTAADTAVYYCPRG
PTMIRGVIDSWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKRVEPKSCDKTHTCPPCPAPEAEGAPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPSSIEKTISKAKGQPREP
QVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPG
K
(LC of Antibody A)
SEQ ID NO: 5
EIVLTQSPATLSLSPGERATLSCRASQSVRSYLAWYQQKPGQAPRLLIYD
ASNRATGIPARFSGSGSGTDFTLTISSLEPEDFAVYYCQQRSNWPPTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
(DNA of HC of Antibody A)
SEQ ID NO: 6
CAGCTGCAGGTGCAGGAGTCGGGCCCAGGACTGGTGAAGCCTTCGGAGAC
CCTGTCCCTCACCTGCACTGTCTCTGGTGGCTCCATCAGCAACAGTTATT
TCTCCTGGGGCTGGATCCGCCAGCCCCCAGGGAAGGGACTGGAGTGGATT
GGGAGTTTCTATTATGGTGAAAAAACCTACTACAACCCGTCCCTCAAGAG
CCGAGCCACCATATCCATTGACACGTCCAAGAGCCAGTTCTCCCTGAAGC
TGAGCTCTGTGACCGCCGCAGACACGGCTGTGTATTACTGTCCGAGAGGG
CCTACTATGATTCGGGGAGTTATAGACTCCTGGGGCCAGGGAACCCTGGT
CACCGTCTCCTCAGCTAGCACCAAGGGCCCATCGGTCTTCCCCCTGGCAC
CCTCCTCCAAGAGCACCTCTGGGGGCACAGCGGCCCTGGGCTGCCTGGTC
AAGGACTACTTCCCCGAACCGGTGACGGTGTCGTGGAACTCAGGCGCACT
GACCAGCGGCGTGCACACCTTCCCGGCTGTCCTACAGTCCTCAGGACTCT
ACTCCCTCAGCAGCGTGGTGACCGTGCCCTCCAGCAGCTTGGGCACCCAG
ACCTACATCTGCAACGTGAATCACAAGCCCAGCAACACCAAGGTGGACAA
GAGAGTTGAGCCCAAATCTTGTGACAAAACTCACACATGCCCACCGTGCC
CAGCACCTGAAGCCGAGGGGGCACCGTCAGTCTTCCTCTTCCCCCCAAAA
CCCAAGGACACCCTCATGATCTCCCGGACCCCTGAGGTCACATGCGTGGT
GGTGGACGTGAGCCACGAAGACCCTGAGGTCAAGTTCAACTGGTATGTGG
ACGGCGTGGAGGTGCATAATGCCAAGACAAAGCCGCGGGAGGAGCAGTAC
AACAGCACGTACCGTGTGGTCAGCGTCCTCACCGTCCTGCACCAAGACTG
GCTGAATGGCAAGGAGTACAAGTGCAAGGTCTCCAACAAAGCCCTCCCAT
CCTCCATCGAGAAAACCATCTCCAAAGCCAAAGGGCAGCCCCGAGAACCA
CAGGTGTACACCCTGCCCCCATCCCGGGAGGAGATGACCAAGAACCAAGT
CAGCCTGACCTGCCTGGTCAAAGGCTTCTATCCCAGCGACATCGCCGTGG
AGTGGGAGAGCAATGGGCAGCCGGAGAACAACTACAAGACCACGCCTCCC
GTGCTGGACTCCGACGGCTCCTTCTTCCTCTATTCCAAGCTCACCGTGGA
CAAGAGCAGGTGGCAGCAGGGGAACGTCTTCTCATGCTCCGTGATGCATG
AGGCTCTGCACAACCACTACACGCAGAAGAGCCTCTCCCTGTCTCCGGGC
AAA
(DNA of LC of Antibody A)
SEQ ID NO: 7
GAAATTGTGTTGACGCAGTCTCCAGCCACCCTGTCTTTGTCTCCAGGGGA
AAGAGCCACCCTCTCCTGCAGGGCCAGTCAGAGTGTTCGCAGCTACTTAG
CCTGGTACCAACAGAAACCTGGCCAGGCTCCCAGGCTCCTCATCTATGAT
GCATCCAACAGGGCCACTGGCATCCCAGCCAGGTTCAGTGGCAGTGGGTC
TGGGACAGACTTCACTCTCACCATCAGCAGCCTAGAGCCTGAAGATTTTG
CAGTTTATTACTGTCAGCAGCGTAGCAACTGGCCTCCGACGTTCGGCCAA
GGGACCAAGGTGGAAATCAAACGAACTGTGGCTGCACCATCTGTCTTCAT
CTTCCCGCCATCTGATGAGCAGTTGAAATCTGGAACTGCCTCTGTTGTGT
GCCTGCTGAATAACTTCTATCCCAGAGAGGCCAAAGTACAGTGGAAGGTG
GATAACGCCCTCCAATCGGGTAACTCCCAGGAGAGTGTCACAGAGCAGGA
CAGCAAGGACAGCACCTACAGCCTCAGCAGCACCCTGACGCTGAGCAAAG
CAGACTACGAGAAACACAAAGTCTACGCCTGCGAAGTCACCCATCAGGGC
CTGAGCTCGCCCGTCACAAAGAGCTTCAACAGGGGAGAGTGT
(HC of Antibody B)
SEQ ID NO: 8
QLQVQESGPGLVKPSETLSLTCTVSGGSISNSYFSWGWIRQPPGKGLEWI
GSFYYGEKTYYNPSLKSRATISIDTSKSQFSLKLSSVTAADTAVYYCPRG
PTMIRGVIDSWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKRVEPKSCDKTHTCPPCPAPEAEGAPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPX1X2IEKTISKAKGQPR
EPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLS
PGK
Wherein X1 is S or A; and X2 is S or P.
(HC of Antibody C)
SEQ ID NO: 9
QVQLVQSGAEVKKPGSSVKVSCKASGGTFSSYAISWVRQAPGQGLEWMGG
IIPIFGTANYAQKFQGRVTITADKSTSTAYMELSSLRSEDTAVYYCARSP
DYSPYYYYGMDVWGQGTTVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKRVEPKSCDKTHTCPPCPAPEAEGAPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPSSIEKTISKAKGQPR
EPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLS
PGK
(LC of Antibody C)
SEQ ID NO: 10
QSVLTQPPSASGTPGQRVTISCSGSSSNIGSNTVNWYQQLPGTAPKLLIY
GNSNRPSGVPDRFSGSKSGTSASLAISGLQSEDEADYYCQSYDSSLSGSV
FGGGIKLTVLGQPKAAPSVTLFPPSSEELQANKATLVCLISDFYPGAVTV
AWKADSSPVKAGVETTTPSKQSNNKYAASSYLSLTPEQWKSHRSYSCQVT
HEGSTVEKTVAPAECS
(HC of Antibody D)
SEQ ID NO: 11
QDQLVESGGGVVQPGRSLRLSCAASGFTESSYGMHWVRQAPGEGLEWVAV
IWYDGSNKYYADSVKGRETISRDNSKNTLYLQMNSLRAEDTAVYYCARGD
YEVDYGMDVWGQGTTVTVASASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKRVEPKSCDKTHTCPPCPAPELLGGPSVFLEPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFELYSKLTVDKSRWQQGNVESCSVMHEALHNHYTQKSLSLSPGK
(LC of Antibody D)
SEQ ID NO: 12
AIQLTQSPSSLSASVGDRVTITCRASQGISNALAWYQQKPGKAPKLLIYD
ASSLESGVPSRFSGSGSGTDFTLTISSLQPEDFATYYCQQFNSYPWTFGQ
GTKVEIKRTVAAPSVFIFPPSDEQLKSGTASVVCLLNNEYPREAKVQWKV
DNALQSGNSQESVTEQDSKDSTYSLSSTLTLSKADYEKHKVYACEVTHQG
LSSPVTKSFNRGEC

We claim:

1. An antibody comprising a light chain (LC) and a heavy chain (HC), wherein the amino acid sequence of the LC is SEQ ID NO: 5, and the amino acid sequence of the HC is SEQ ID NO: 8.

2. The antibody of claim 1, wherein the amino acid sequence of the HC is SEQ ID NO: 4.

3. The antibody of claim 1, comprising two light chains and two heavy chains, wherein the amino acid sequence of each LC is SEQ ID NO: 5, and the amino acid sequence of each HC is SEQ ID NO: 8.

4. The antibody of claim 3, wherein the amino acid sequence of each HC is SEQ ID NO: 4.

5. A mammalian cell comprising a DNA molecule comprising a polynucleotide sequence encoding a polypeptide having the amino acid sequence of SEQ ID NO: 5 and a polynucleotide sequence encoding a polypeptide having the amino acid sequence of SEQ ID NO: 4, wherein the cell is capable of expressing an antibody comprising a light chain having the amino acid sequence of SEQ ID NO: 5 and a heavy chain having the amino acid sequence of SEQ ID NO: 4.

6. A mammalian cell comprising a first DNA molecule and a second DNA molecule, wherein the first DNA molecule comprises a polynucleotide sequence encoding a polypeptide having the amino acid sequence of SEQ ID NO: 5, and wherein the second DNA molecule comprises a polynucleotide sequence encoding a polypeptide having the amino acid sequence of SEQ ID NO: 4, wherein the cell is capable of expressing an antibody comprising a light chain having the amino acid sequence of SEQ ID NO: 5 and a heavy chain having the amino acid sequence of SEQ ID NO: 4.

7. A process for producing an antibody comprising cultivating the mammalian cell of claim 5 under conditions such that the antibody is expressed, and recovering the expressed antibody.

8. A process for producing an antibody comprising cultivating the mammalian cell of claim 6 under conditions such that the antibody is expressed, and recovering the expressed antibody.

9. An antibody produced by cultivating a mammalian cell comprising a DNA molecule comprising a polynucleotide sequence encoding a polypeptide having an amino acid sequence of SEQ ID NO: 5 and a polynucleotide sequence encoding a polypeptide having an amino acid sequence of SEQ ID NO: 4 under conditions such that the antibody is expressed, and recovering the expressed antibody.

10. An antibody produced by cultivating a mammalian cell comprising a first DNA molecule and a second DNA molecule, wherein the first DNA molecule comprises a polynucleotide sequence encoding a polypeptide having the amino acid sequence of SEQ ID NO: 5, and wherein the second DNA molecule comprises a polynucleotide sequence encoding a polypeptide having the amino acid sequence of SEQ ID NO: 4 under conditions such that the antibody is expressed, and recovering the expressed antibody.

11. A pharmaceutical composition, comprising the antibody of claim 1 and an acceptable carrier, diluent, or excipient.

12. A method of treating fibrosis, comprising administering to a patient in need thereof, an effective amount of the antibody of claim 1.

13. A method of treating cancer, comprising administering to a patient in need thereof, an effective amount of the antibody of claim 1.

14. The method of claim 13, wherein the cancer is breast cancer, colon cancer, gastric cancer, glioblastoma, head and neck cancer, hepatocellular carcinoma, non-small cell lung cancer (NSCLC), small cell lung cancer (SCLC), melanoma, myelodysplastic syndrome, pancreatic cancer, prostate cancer, or renal cancer.

15. The method of claim 14, further comprising administering simultaneously, separately, or sequentially one or more anti-tumor agents, selected from the group consisting of platinum-based antineoplastic drugs, taxanes, fluoropyrimidines, enzalutamide, abiraterone, sorafenib, IMC-GP100, abemaciclib, and ramucirumab.

16. The method of claim 14, further comprising administering simultaneously, separately, or sequentially an anti-PDL1 antibody comprising two light chains and two heavy chains, wherein the amino acid sequence of each light chain is SEQ ID NO: 10, and the amino acid sequence of each heavy chain is SEQ ID NO: 9.

17. The method of claim 14, further comprising administering simultaneously, separately, or sequentially an anti-CSF-1R antibody comprising two light chains and two heavy chains, wherein the amino acid sequence of each light chain is SEQ ID NO: 12, and the amino acid sequence of each heavy chain is SEQ ID NO: 11.


SEQUENCE LISTING (12 sequences)

SEQ ID NO: 1 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 2 - protein, 121 aa, Artificial Sequence
  synthetic construct
QLQVQESGPGLVKPSETLSLTCTVSGGSISNSYFSWGWIRQPPGKGLEWIGSFYYGEKTYYNPSLKSRATISIDTSKSQFSLKLSSVTAADTAVYYCPRGPTMIRGVIDSWGQGTLVTVSS

SEQ ID NO: 3 - protein, 107 aa, Artificial Sequence
  synthetic construct
EIVLTQSPATLSLSPGERATLSCRASQSVRSYLAWYQQKPGQAPRLLIYDASNRATGIPARFSGSGSGTDFTLTISSLEPEDFAVYYCQQRSNWPPTFGQGTKVEIK

SEQ ID NO: 4 - protein, 451 aa, Artificial Sequence
  synthetic construct
QLQVQESGPGLVKPSETLSLTCTVSGGSISNSYFSWGWIRQPPGKGLEWIGSFYYGEKTYYNPSLKSRATISIDTSKSQFSLKLSSVTAADTAVYYCPRGPTMIRGVIDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 214 aa, Artificial Sequence
  synthetic construct
EIVLTQSPATLSLSPGERATLSCRASQSVRSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - DNA, 1353 bp, Artificial Sequence
  synthetic construct
cagctgcaggtgcaggagtcgggcccaggactggtgaagccttcggagaccctgtccctcacctgcactgtctctggtggctccatcagcaacagttatttctcctggggctggatccgccagcccccagggaagggactggagtggattgggagtttctattatggtgaaaaaacctactacaacccgtccctcaagagccgagccaccatatccattgacacgtccaagagccagttctccctgaagctgagctctgtgaccgccgcagacacggctgtgtattactgtccgagagggcctactatgattcggggagttatagactcctggggccagggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgcactgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgagggggcaccgtcagtcttcctcttccccccaaaacccaaggacaccctcatgatctcccggacccctgaggtcacatgcgtggtggtggacgtgagccacgaagaccctgaggtcaagttcaactggtatgtggacggcgtggaggtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcccatcctccatcgagaaaaccatctccaaagccaaagggcagccccgagaaccacaggtgtacaccctgcccccatcccgggaggagatgaccaagaaccaagtcagcctgacctgcctggtcaaaggcttctatcccagcgacatcgccgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctccgggcaaa

SEQ ID NO: 7 - DNA, 642 bp, Artificial Sequence
  synthetic construct
gaaattgtgttgacgcagtctccagccaccctgtctttgtctccaggggaaagagccaccctctcctgcagggccagtcagagtgttcgcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccgacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 8 - protein, 451 aa, Artificial Sequence
  synthetic construct
  MISC_FEATURE (334)..(334): Xaa at position 334 is Ser or Ala
  MISC_FEATURE (335)..(335): Xaa at position 335 is Ser or Pro
QLQVQESGPGLVKPSETLSLTCTVSGGSISNSYFSWGWIRQPPGKGLEWIGSFYYGEKTYYNPSLKSRATISIDTSKSQFSLKLSSVTAADTAVYYCPRGPTMIRGVIDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPXXIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 453 aa, Artificial Sequence
  synthetic construct
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARSPDYSPYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 216 aa, Artificial Sequence
  synthetic construct
QSVLTQPPSASGTPGQRVTISCSGSSSNIGSNTVNWYQQLPGTAPKLLIYGNSNRPSGVPDRFSGSKSGTSASLAISGLQSEDEADYYCQSYDSSLSGSVFGGGIKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPAECS

SEQ ID NO: 11 - protein, 450 aa, Artificial Sequence
  synthetic construct
QDQLVESGGGVVQPGRSLRLSCAASGFTFSSYGMHWVRQAPGEGLEWVAVIWYDGSNKYYADSVKGRFTISRDNSKNTLYLQMNSLRAEDTAVYYCARGDYEVDYGMDVWGQGTTVTVASASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 214 aa, Artificial Sequence
  synthetic construct
AIQLTQSPSSLSASVGDRVTITCRASQGISNALAWYQQKPGKAPKLLIYDASSLESGVPSRFSGSGSGTDFTLTISSLQPEDFATYYCQQFNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC